(19) United States
(12) Patent Application Publication
(10) Pub. No.: US 2026/0130792 A1
(43) Pub. Date: May 14, 2026

(54) METHODS AND DEVICES FOR TREATING RECTAL HEMORRHOIDS

(71) Applicant: Correct`em Corporation, Derry, NH (US)

(72) Inventor: Rachel Anne MacNeill, Derry, NH (US)

(21) Appl. No.: 19/387,033

(22) Filed: Nov. 12, 2025

Related U.S. Application Data
(60) Provisional application No. 63/719,593, filed on Nov. 12, 2024.

(51) Int. Cl. A61F 7/12; A61F 7/00

(52) U.S. Cl. CPC A61F 7/12; A61F 2007/0028

(57) ABSTRACT

The disclosure provides a hemorrhoid splinting device intended for insertion into the anorectal cavity of a human. The device includes an elongated, fusiform body with a middle portion of larger diameter that tapers to smaller-diameter distal and proximal ends, where the distal end is rounded and the proximal end includes a handle. The device may feature surface recesses for administering medication, and/or an internal cavity for substances that retain coolant or heat. Another embodiment of the device includes an outer perforated skin layer for controlled release of therapeutic agents. Medications, such as hydrocortisone or witch hazel, may be applied to the recesses or positioned between the body and skin layer. Methods of treating hemorrhoids include inserting the device and maintaining the device inserted for a period of time.

### FIELD

The present invention relates to medical devices and methods for the treatment and management of hemorrhoids.

### BACKGROUND

Rectal hemorrhoids affect greater than 10 million Americans each year. The management of hemorrhoids is a significant concern in both primary care, gastroenterology, and pelvic health. Hemorrhoidal prolapse and its associated symptoms—pain, swelling, itching, and bleeding—cause discomfort, pain, and functional limitation for many people. Conventional strategies, including dietary fiber supplementation, sitz baths, and over-the-counter topical applications, are widely used but often fail to provide lasting relief. Many individuals continue to experience persistent symptoms, underscoring the need for more effective and user-friendly solutions that address both tissue displacement and discomfort.

Non-surgical treatments have traditionally focused on symptom management rather than the anatomical causes of prolapse. Often hemorrhoids require manual reduction with a finger, which is impractical for most and which typically results in only transient maintenance of the reduction of the prolapsed tissue. Topical agents and suppositories may offer temporary relief, but their effects are limited by rapid absorption, expulsion, and the lack of mechanical support. Patient reluctance to seek care for sensitive anorectal issues can further delay effective treatment. This leaves a substantial gap between symptom-oriented therapies and the need for prolonged mechanical stabilization to maintain tissue reduction. The absence of a dedicated device for maintaining prolapsed hemorrhoids in a reduced position highlights the need for a streamlined, non-surgical approach specifically tailored for anorectal support.

### SUMMARY

In one aspect, this disclosure provides a hemorrhoid splinting device sized and shaped for insertion into an anorectal cavity of a human. The device includes an elongated body having a generally fusiform shape with a middle portion having a first diameter, a distal end having a second diameter smaller than the first diameter with the distal end rounded, and a proximal end having a third diameter smaller than the first diameter. The elongated body tapers from the middle portion to the distal end. The surface of the elongated body comprises one or more recesses. A handle is attached to the proximal end of the elongated body. In some embodiments, the handle is attached to the proximal end via a neck portion.

In some embodiments, the device further comprises a cavity inside the elongated body. In some embodiments, the cavity contains water, a coolant gel, or a coolant fluid. In some embodiments, the cavity contains a heat-retention substance. In some embodiments, the cavity is sealed. In some embodiments, the cavity comprises a port configured to receive fluid from outside of the device.

In some embodiments, the device further comprises a medication. In some embodiments, the one or more recesses contain the medication. In some embodiments, the medication comprises a topical medication. In some embodiments, the medication comprises hydrocortisone. In some embodiments, the medication comprises witch hazel. In some embodiments, the medication comprises a local anesthetic. In some embodiments, the medication comprises lidocaine. In some embodiments, the medication comprises a vasoconstrictor. In some embodiments, the medication comprises phenylephrine hydrochloride.

In some embodiments, the device comprises silicone.

In another aspect, this disclosure provides a hemorrhoid splinting device, sized and shaped for insertion into an anorectal cavity of a human, and comprising an elongated body having a generally fusiform shape, a middle portion having a first diameter, a distal end having a second diameter smaller than the first diameter with the distal end rounded, a proximal end having a third diameter smaller than the first diameter, and a handle attached to the proximal end. The device further includes an outer skin layer that wraps around a majority of the core elongated body and comprises one or more perforations. In some embodiments, the handle is attached to the proximal end via a neck portion.

In some embodiments of the device, a cavity is inside the elongated body. In some embodiments of the device, the cavity contains water, a coolant gel, or a coolant fluid. In some embodiments of the device, the cavity contains a heat-retention substance. In some embodiments of the device, the cavity is sealed. In some embodiments of the device, the cavity comprises a port configured to receive fluid from outside of the device.

In some embodiments of the device, a medication is positioned between the elongated body and the outer skin layer. In some embodiments of the device, the perforations of the outer skin layer are configured to allow medication to flow from the device to the surrounding tissue

when inserted into an anorectal cavity. In some embodiments of the device, the medication comprises a topical medication. In some embodiments of the device, the medication comprises hydrocortisone. In some embodiments, the medication comprises witch hazel. In some embodiments, the medication comprises a local anesthetic. In some embodiments, the medication comprises lidocaine.

In some embodiments of the treatment device, the device comprises silicone.

Another aspect of this disclosure is directed to a method of treating a hemorrhoid in a human. The method includes providing a hemorrhoid splinting device of this disclosure. In some embodiments, device includes an elongated body having a generally fusiform shape with a middle portion having a first diameter, a distal end having a second diameter smaller than the first diameter with the distal end rounded, and a proximal end having a third diameter smaller than the first diameter. The elongated body tapers from the middle portion to the distal end. The surface of the elongated body comprises one or more recesses. A handle is attached to the proximal end of the elongated body. In some embodiments, the handle is attached to the proximal end via a neck portion. The method also comprises inserting the hemorrhoid splinting device into an anorectal cavity of the human having a hemorrhoid and maintaining the device inserted in the anorectal cavity for a time period sufficient to ameliorate one or more symptoms of the hemorrhoid.

In some embodiments of the method, a medication is applied to the one or more recesses. In some embodiments of the method, the medication comprises a topical medication. In some embodiments of the method, the medication comprises hydrocortisone. In some embodiments of the method, the medication comprises witch hazel. In some embodiments of the method, the medication comprises a local anesthetic. In some embodiments of the method, the medication comprises lidocaine.

In some embodiments of the method, the device comprises a coolant substance and the method further comprises cooling the device prior to inserting the device.

In some embodiments of the method, the device comprises silicone.

### BRIEF DESCRIPTION OF THE FIGURES

FIG. 1 is a perspective view of an embodiment of the hemorrhoid splinting device of this disclosure.

### DETAILED DESCRIPTION

This disclosure pertains to medical devices and methods for treating and managing hemorrhoids, specifically through the use of a hemorrhoid splinting device designed for insertion into the anorectal cavity to provide mechanical support and to deliver a therapeutic agent. The described embodiments address both anatomical and symptomatic aspects of hemorrhoids, offering a non-surgical, user-friendly solution.

As used herein, the articles “a” and “an” mean one or more than one, unless context dictates otherwise. As used herein, the terms “subject” and “patient” are used interchangeably and refer to a human.

The hemorrhoid splinting device of this disclosure features an elongated, fusiform body with a middle portion having a first diameter. In some embodiments, the device tapers to a distal end having a second diameter that is smaller than the first diameter. The device also comprises a proximal end having a third diameter. In some embodiments, the diameter of the distal end is smaller than the diameter of the proximal end. In some embodiments, the diameter of the distal end is the same as the diameter of the proximal end. In some embodiments, the diameter of the distal end is larger than the diameter of the proximal end. In some embodiments, the distal end is rounded for ease of insertion. The proximal end is attached to a handle for user-friendly operation. In some embodiments, the proximal end is connected to a handle via a neck portion. In some embodiments, the handle has a T-shape, an L-shape, a crescent shape, a curved rod shape, or is shaped like a ball or a flat disc. The handle may be flexible.

In some embodiments, the surface of the device includes one or more recesses for delivering topical medications, such as hydrocortisone or witch hazel, directly to the affected tissue. The recesses can have different shapes or all be the same shape. The recesses can have any suitable shape that allows medication to flow. In some embodiments, the recesses have a hexagonal shape. In some embodiments, the recesses have a pentagonal shape. In some embodiments, the recesses have a triangular shape. In some embodiments, the recesses are shallow, concave spherical depressions. In some embodiments, the recesses are another geometric shape that allows the device to retain medication while allowing the medication to flow over time.

In use, the device is inserted into an anorectal cavity of a human and maintained in place for a duration sufficient to alleviate symptoms, such as inflammation, discomfort, or prolapsed tissue. After insertion, the handle of the device remains outside of the patient's body. In some embodiments, the patient wears the device for a specified number of minutes or hours. In some embodiments, the patient wears the device while sleeping. When the device has medication loaded into one or more recesses or in between a skin layer and the device core, the medication seeps from the one or more recesses to the surrounding tissue or from in between the skin layer and device core to the surrounding tissue, including to one on more hemorrhoids in need of treatment. Different medications may seep or ooze from the device depending on their viscosities.

In some embodiments, the device is cooled in a freezer or refrigerator prior to insertion. In some embodiments, the device is warmed prior to insertion. In some embodiments, medication is applied to the recesses prior to insertion. The one or more recesses allow for controlled medication release. Variations in the configuration of the device, such as the inclusion of a fluid-fill reservoir with water, a coolant gel, a coolant fluid, or a heat retention gel further enhance adaptability to individual treatment requirements.

When inserted, the device splints against one or more hemorrhoids. Over time, while the patient wears the inserted device, the device mechcanically stabilizes the prolapsed tissue. This splinting can redirect prolapsed tissue back into place and can inhibit further prolapse. While the hemorrhoid is braced by the splinting device, the prolapsed tissue can reduce. The device further enhances tissue healing and resolution of hemorrhoids in embodiments of the device that include thermotherapy and/or delivery of medication.

This integrated approach offers a comprehensive, non-surgical solution for the sustained management of hemorrhoids. The device provides mechanical splinting to support repair of prolapsed tissue. In some embodiments, the device combines mechanical support with delivery of a medication to further promote tissue healing. The device of this disclosure addresses both anatomical and symptomatic aspects of hemorrhoids.

In some embodiments, the device may be manufactured from a range of biocompatible materials, including silicone for flexibility and comfort, rubber for enhanced grip and resilience, and plastic for cost-effective production. In another embodiment, the choice of material may be tailored to patient preference, intended use (disposable or reusable), and manufacturing requirements. In some embodiments, the device comprises silicone. In certain embodiments, the device is made entirely from silicone. In some embodiments, the device is made from a combination of silicone and plastic.

In some embodiments, the hemorrhoid splinting device is disposable. In some embodiments, the disposable hemorrhoid splinting device is fabricated from biodegradable material, such as plant-based polymer, or from medical-grade plastic.

In some embodiments, the device is pre-loaded with a topical medication, such as hydrocortisone or witch hazel, within its surface recesses or between the core elongated body and the skin layer. In some embodiments more than one topical medication may be applied to different parts of the device. After single use, the device may be safely discarded, minimizing environmental impact and reducing the risk of cross-contamination. In some embodiments, the device may be sanitized and re-used. In some embodiments, sanitization may be accomplished by washing the device, boiling the device, subjecting the device to UV radiation, or a combination. Washing the device may be via soap and water, a dishwasher, or sanitizing wipes.

In some embodiments, the device is constructed with a central core and an external sheath layer, wherein the sheath layer contains multiple perforations or holes designed to gradually release topical medication. In another embodiment, the device is configured in a syringe-style form, allowing the user to dispense medication directly to the affected tissue during insertion. In such embodiments, the handle is connected to a piston that is fluidically connected to one or more tubes. The outlets of the one or more tubes are situated at or near perforations on the device. The device is pre-loaded with medication, including medication in the tubes. When the handle is pressed, the handle actuates the piston, thereby pushing into the one or more tubes and causing medication to be dispensed from the outlets and into and through the perforations. The handle and piston need not move a lot to cause the dispensing. After medication is pushed through the tubes, the medication is further disbursed from the inner cavity of the device through the outlets via the natural movement of the muscles of the distal gastrointestinal tract during wear. In some embodiments, one medication is disbursed through the outlets and another medication is applied directly to recesses on the surface of the device.

In some embodiments, a hemorrhoid splinting device is equipped with one or more sensors capable of tracking pressure exerted by the device, temperature at the site of application, and other physiological signs, such as indicators of constipation or inflammation. Sensors may also track device duration and frequency of wear. A sensor may include a transmitter for transmitting collected data via Bluetooth or another short range communication standard such as Near Field Communication (NFC) or via Wi-Fi. Data collected by the device may be transmitted to a mobile application installed on a mobile device for patient monitoring and feedback to healthcare providers, and to provide a platform for tracking variables as described above.

In some embodiments, the device incorporates thermotherapy capability, allowing the user to apply heat or cold to the affected area for additional symptom relief. In some embodiments, the device comprises a reservoir for storing a thermotherapy liquid or gel. In some embodiments, the thermotherapy liquid or gel is water, a coolant gel, a coolant fluid, or a heat-retention gel or fluid. When cool treatment is desired, the device can be refrigerated or stored in a freezer prior to use. The thermotherapy liquid or gel provides cooling relief when the device is inserted. Alternatively, if heat is desired, the device may be warmed. In some embodiments, the device may be warmed in a microwave or may be warmed in a pot of warm water prior to insertion. The heat retention liquid or gel retains the heat and provides warming relief when inserted.

In another embodiment, a vibrating mechanism may be included to ease discomfort during insertion and removal, improving patient compliance and comfort. In some embodiments, the device comprises an eccentric rotating mass motor or a linear resonant actuator to create vibration. In some embodiments, vibration is activated by the press of a discrete button on the device or via a connected application installed on a mobile device. The button to activate vibration may be on the handle of the device and may be operated in a standard on/off manner or may be set to a timer so that, after turning vibration on, vibration shuts off after a preset amount of time. The vibration may be activated further at the conclusion of wear, to alert the user to recommended removal. Any button mechanism is sealed to prevent contamination and maximize cleanliness.

In some embodiments, a device is provided with a wider external anchor or flange, specifically shaped to cover and support external hemorrhoidal tissue. In another embodiment, the anchor may be available in different sizes and shapes to address varying anatomical needs, to ensure secure placement, or for comfort.

In some embodiments, the device may include a self-inflating feature, such as an internal bladder or chamber that can be expanded by the user to achieve a custom fit and apply adjustable pressure to the affected area. This embodiment allows for personalized treatment and improved mechanical stabilization. In some embodiments, inflation is activated by the press of a discrete button on the device or via a connected application installed on a mobile device. In some embodiments, the amount of inflation can be controlled by Bluetooth via an application installed on a mobile device.

In some embodiments, the hemorrhoid splinting device may be offered in a range of sizes and general shapes to accommodate different patient anatomies and severities of hemorrhoidal disease. The device is anchored in the body via natural resting tone of the anal sphincter muscles, and device size is informed by both requirements to optimize tissue contact and to maximize comfort with wear. The body of the device from distal tip to proximal taper may range in length from about 0.5 to about 8 cm. The distal portion, middle portion, and proximal portion range in diameter from about 0.5 mm to about 1 cm. In various embodiments, the distal portion, middle portion, and proximal portion have diameters ranging up to about 2 cm. The maximum diameter of the middle portion should not exceed 6 cm. The neck of the device connecting the proximal taper of the body to the handle may range in length from about 0.5 to about 6 cm. Thus, the length of the device from distal tip to handle may range from about 1 cm to about 14 cm. The maximal diameter of the neck may not exceed 4 cm. In another embodiment, external anchors may also be provided in various sizes and configurations to specifically address external hemorrhoids.

The hemorrhoid splinting device can be made through a variety of methods. In some embodiments, the hemorrhoid splinting device is made from a mold. In other embodiments, the hemorrhoid splinting device is made from one uniform piece. In some embodiments, the hemorrhoid splinting device is cast-poured. In some embodiments, particularly where the hemorrhoid splinting device comprises plastic, the hemorrhoid splinting device is manufactured by roto-molding, rotational molding, injection molding, plastic extrusion molding, blow molding, vacuum molding, or by other suitable molding methods. In certain embodiments, injection molding may be performed using a split or multi-part mold to form the desired geometry of the hemorrhoid splinting device. In some embodiments, particularly where the hemorrhoid splinting device comprises silicone or another elastomeric material, the device may be produced using liquid silicone rubber (LSR) injection molding, compression molding, or casting into a mold, followed by curing to achieve the desired flexibility and resilience. In further embodiments, the hemorrhoid splinting device is constructed from two or more separately constructed components and then joined together. In further embodiments, the hemorrhoid splinting device is produced by 3D printing or additive manufacturing, or any process in which material or materials are joined or solidified under computer control to create a three-dimensional object. In some embodiments, rapid prototyping is used to produce the hemorrhoid splinting device.

This disclosure also provides methods of treating hemorrhoids in a patient comprising providing an appropriately sized device of this disclosure; inserting the device into an anorectal cavity of a patient, thereby providing a manual splint for prolapsing tissue; and maintaining the device inserted in the anorectal cavity a period sufficient to ameliorate one or more symptoms of a hemorrhoid. In some embodiments, the period of time is minutes or hours. This disclosure also provides methods of splinting a hemorrhoid in a patient comprising providing an appropriately sized device of this disclosure; inserting the device into an anorectal cavity of a patient, wherein the device contacts a hemorrhoid of the patient, thereby splinting the hemorrhoid; and maintaining the device inserted in the anorectal cavity a period sufficient to ameliorate one or more symptoms of the hemorrhoid.

In some embodiments, prior to insertion, the device is pre-cooled. In some embodiments, prior to insertion, the device is warmed. In some embodiments, medication is applied to the device prior to insertion. In some embodiments, medication is applied to recesses on the surface of the device. In some embodiments, medication is applied between a core elongated body of the device and an outer skin layer comprising perforations.

In some embodiments, physiological conditions are monitored via sensors on the device, which transmit data to a mobile application installed on a mobile device via Bluetooth, NFC, or Wi-Fi. In some embodiments, the method comprises adjusting fit through an inflatable bladder contained in the device or utilizing vibration for pain management, or both. In some methods, the device is inserted and removed via an external handle. In some embodiments, the device may be sanitized and re-used. In some embodiments, sanitization may be accomplished by washing the device, boiling the device, subjecting the device to UV radiation, or a combination.

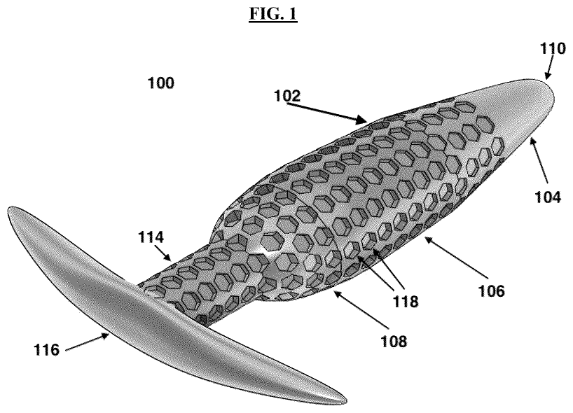

Referring to FIG. 1, hemorrhoid splinting device 100 comprises elongated body 102. Elongated body 102 comprises distal end 104. In some embodiments, distal end 104 comprises rounded end 110 for ease of insertion. Elongated body 102 also comprises middle portion 106. Elongated body 102 tapers from middle portion 106 to distal end 104. Elongated body 102 also comprises proximal end 108. Elongated body 102 also tapers from middle portion 106 to proximal end 108.

Hemorrhoid splinting device 100 comprises handle 116, which allows the user to insert and retrieve the device. As shown in FIG. 1, in some embodiments, handle 116 is connected to proximal end 108 via neck 114. Neck 114 is narrower in diameter than elongated proximal end 108.

### EXAMPLES

The following examples illustrate various embodiments and methods of use for the hemorrhoid splinting device described herein. These examples are provided to facilitate understanding of the invention and are not intended to limit its scope.

### Example 1: Disposable, Pre-Filled Device With Recesses

In one example, the hemorrhoid splinting device is disposable and pre-filled with a medication. The disposable hemorrhoid splinting device is fabricated from biodegradable material, such as plant-based polymer, or from medical-grade plastic. The device is pre-filled with a topical medication, such as hydrocortisone, witch hazel, or lidocaine, on recesses on the surface of the device. After single use, the device may be safely discarded.

### Example 2: Re-Usable Splinting and Cryotherapy Only Device

In one example, the hemorrhoid splinting device is made of a reusable, washable material with a hollow core filled with material that may be cooled in the refrigerator or freezer prior to wear. The device may be worn on its own, without topical medication, for the benefit of mechanical splinting and cryotherapy. The device may further comprise a sensor with Bluetooth capabilities and/or an inflating mechanism for modulating splinting.

### Example 3: Re-Usable Splinting, Cryotherapy and Topical Device

In one example, the hemorrhoid splinting device is made of a reusable, washable material with a hollow core filled with material that may be cooled in the refrigerator or freezer prior to wear. Prior to use, a topical medication, such as hydrocortisone, witch hazel, or lidocaine, is added to surface recesses on the device or to a space within the device. The device may further comprise a sensor with Bluetooth capabilities and/or an inflating mechanism for modulating splinting.

### Example 4: Disposable, Pre-Filled Device With Perforations

In one example, the hemorrhoid splinting device is disposable and pre-filled with a medication. The disposable hemorrhoid splinting device is fabricated from biodegradable material, such as plant-based polymer, or from medical-grade plastic. The device is pre-filled with a topical medication, such as hydrocortisone, witch hazel, or lidocaine, beneath a skin layer that comprises perforations. The skin layer wraps around a substantial portion of the elongated body. After insertion, this topical is then dispersed via perforations to the affected tissue. After single use, the device may be safely discarded.

1. A hemorrhoid splinting device sized and shaped to be inserted into an anorectal cavity of a human, comprising
an elongated body having a generally fusiform shape comprising;
a middle portion having a first diameter;
a distal end having a second diameter that is smaller than the first diameter,
wherein the distal end is rounded;
a proximal end having a third diameter that is smaller than the first diameter,
wherein the elongated body tapers from the middle portion to the distal end; and
a surface comprising one or more recesses; and
a handle attached to the proximal end of the elongated body.

2. The hemorrhoid splinting device of claim 1, wherein the handle is attached to the proximal end via a neck portion.

3. The hemorrhoid splinting device of claim 1, further comprising a cavity inside the elongated body.

4. The hemorrhoid splinting device of claim 2, wherein the cavity contains water, a coolant gel, or a coolant fluid.

5. The hemorrhoid splinting device of claim 2, wherein the cavity contains a heat-retention substance.

6. The hemorrhoid splinting device of claim 2, wherein the cavity is sealed.

7. The hemorrhoid splinting device of claim 2, wherein the cavity comprises a port configured to receive fluid from outside of the device.

8. The hemorrhoid splinting device of claim 1, further comprising a medication.

9. The hemorrhoid splinting device of claim 8, wherein the one or more recesses contain the medication.

10. The hemorrhoid splinting device of claim 8, wherein the medication comprises a topical medication.

11. The hemorrhoid splinting device of claim 8, wherein the medication comprises hydrocortisone.

12. The hemorrhoid splinting device of claim 8, wherein the medication comprises witch hazel.

13. The hemorrhoid splinting device of claim 8, wherein the medication comprises a local anesthetic.

14. The hemorrhoid splinting device of claim 13, wherein the medication comprises lidocaine.

15. The hemorrhoid splinting device of any one of claims 1-14, wherein the device comprises silicone.

16. A hemorrhoid splinting treatment device sized and shaped to be inserted into an anorectal cavity of a human, comprising
an elongated body having a generally fusiform shape;
a middle portion having a first diameter;
a distal end having a second diameter that is smaller than the first diameter, wherein the distal end is rounded;
a proximal end having a third diameter that is smaller than the first diameter;
a handle attached to the proximal end; and
an outer skin layer that wraps around the majority of the elongated body and comprises one or more perforations through the layer.

17. The hemorrhoid splinting device of claim 16, wherein the handle is attached to the proximal end via a neck portion.

18. The hemorrhoid splinting device of claim 16, further comprising a cavity inside the elongated body.

19. The hemorrhoid splinting device of claim 17, wherein the cavity contains water, a coolant gel, or a coolant fluid.

20. The hemorrhoid splinting device of claim 17, wherein the cavity contains a heat-retention substance.

21. The hemorrhoid splinting device of claim 17, wherein the cavity is sealed.

22. The hemorrhoid splinting device of claim 17, wherein the cavity comprises a port configured to receive fluid from outside of the device.

23. The hemorrhoid splinting device of claim 16, further comprising a medication between the elongated body and the outer skin layer.

24. The hemorrhoid splinting device of claim 23, wherein the perforations of the outer skin layer are configured to allow the medication to flow.

25. The hemorrhoid splinting device of claim 24, wherein the medication comprises a topical medication.

26. The hemorrhoid splinting device of claim 24, wherein the medication comprises hydrocortisone.

27. The hemorrhoid splinting device of any one of claims 16 to 26, wherein the device comprises silicone.

28. A method of treating a hemorrhoid in a human, comprising
providing a hemorrhoid splinting device comprising;
an elongated body having a generally fusiform shape comprising;
a middle portion having a first diameter;
a distal end having a second diameter that is smaller than the first diameter, wherein the distal end is rounded;
a proximal end having a third diameter that is smaller than the first diameter,
wherein the elongated body tapers from the middle portion to the distal end;
a surface comprising one or more recesses; and
a handle attached to the proximal end of the elongated body;
inserting the hemorrhoid splinting device into an anorectal cavity of the human so that the handle remains outside of the human;
maintaining the hemorrhoid splinting device inserted in the anorectal cavity for a time period sufficient to ameliorate one or more symptoms of the hemorrhoid.

29. The method of claim 28, further comprising applying a medication to the one or more recesses.

30. The method of claim 29, wherein the medication comprises a topical medication.

31. The method of claim 30, wherein the medication comprises hydrocortisone.

32. The method of claim 28, wherein the medication comprises witch hazel.

33. The method of claim 28, wherein the medication comprises a local anesthetic.

34. The method of claim 33, wherein the medication comprises lidocaine.

35. The method of claim 28, wherein the device comprises a coolant substance and the method further comprising cooling the device prior to inserting the device.

36. The method of any one of claims 28 to 35, wherein the device comprises silicone.
